(19) United States
(12) Patent Application Publication
(10) Pub. No.: US 2003/0040515 A1
(43) Pub. Date: Feb. 27, 2003

(54) Regulation of platelet adhesion and aggregation

(76) Inventors: Philip John Hogg, Randwick (AU); Colin Nicholson Chesterman, Edgecliff (AU)

Correspondence Address: MOLINS AND CO., LEVEL 25 CHIFLEY TOWER, SYDNEY, 2000, AU

(21) Appl. No.: 10/203,335

(22) PCT Filed: Feb. 9, 2001
(86) PCT No.: PCT/AU01/00119

(51) Int. Cl. A61B 17/20; A61K 31/535

(52) U.S. Cl. 514/237.5; 604/22

(57) ABSTRACT

The present invention relates to methods for regulating platelet adhesion and aggregation in a subject. The methods involve administration of analogues or antagonists of thrombospondin-1 (TSP-1).

### TECHNICAL FIELD

The present invention relates generally to the field of blood function and thrombosis and to methods and products useful in the treatment of thrombotic and vascular disorders.

### BACKGROUND ART

Binding of platelets to von Willebrand factor (vWF) in the subendothelium of a damaged blood vessel is the initial step in formation of a haemostatic plug. vWF is also the carrier for pro-coagulant factor VIII protecting it from inactivation by activated protein C and factor Xa in the circulating blood. vWF is synthesized by vascular endothelial cells and megakaryocytes and circulates in blood as a series of multimers containing a variable number of ˜500 kDa homodimers. The largest vWF multimers have a molecular mass of ˜20,000 kDa and are comparable in length to the diameter of a medium platelet (2 μM).

vWF dimers are assembled from pairs of ˜250 kDa polypeptide subunits in the endoplasmic reticulum via disulfide bridges between cysteine residues located in the carboxy terminal regions. Inter-subunit disulfide bonds involve one or three of the Cys residues at positions 2008, 2010 and 2048. Subsequently, multimers are formed by interdimeric disulfide linking of amino terminal domains in a parallel orientation. Inter-dimeric disulfide bonds involve Cys379 and one or more of the Cys residues at positions 459, 462, and 464 (Doug et al., 1994).

Only the large multimeric forms of vWF are haemostatically active (Furlan, 1996). This relates to affinity of vWF for its ligands. Binding of multimeric vWF to collagen occurs with ˜100-fold higher affinity than binding of monomeric vWF fragments. Similarly, large vWF multimers bind to activated platelets with up to 10-fold higher affinity than small multimers, or with ˜100-fold higher affinity than monomeric fragments (reviewed in Furlan, 1996). Large multimers of vWF have considerably higher ristocetin cofactor activity per unit antigen than small multimers (Furlan et al., 1979), and the unusually large vWF multimers secreted by endothelial cells have been shown to be more effective than the largest plasma forms in inducing platelet aggregation under conditions of high fluid shear (Moake et al., 1989). Some thrombotic disorders are characterized by altered vWF multimer size. Thrombotic thrombocytopenic purpura (TTP) is usually associated with unusually large vWF multimers in the blood which are thought to precipitate intravascular platelet clumping (Moake. 1997). Conversely, lower than average multimer size characterizes the bleeding diathesis of type II von Willebrand disease.

### SUMMARY OF THE INVENTION

The present inventors have found that the conditioned medium of cultured macrovascular and microvascular endothelial cells contains an activity which reduces the average multimer size of plasma or purified vWF. The reducing activity is ablated by pretreatment with heat or thiol blocking agents, but not by a range of specific proteinase inhibitors. Reduction in vWF multimer size is associated with formation of new thiols in vWF and there is no evidence for additional proteolytic processing of vWF. This reductase has been isolated and identified as the trimeric glycoprotein, thrombospondin-1 (TSP-1).

The present inventors have therefore made the surprising finding that TSP-1 has an activity that reduces the average multimer size of vWF. This finding has important implications for the development of antiplatelet therapies. One of the early events in the formation of a thrombis is the adhesion of circulating platelets to multimeric vWF molecules bound to the vascular endothelium. This primary adhesion to the matrix activates the platelets, which subsequently secrete several different compounds, some of which attract more platelets to the lumen of the artery and promote aggregation. The ability to interfere with the early event of platelet adhesion to the endothelium by reducing multimeric vWF would provide a significant advantage over alternative antiplatelet therapies that act at the later stages of platelet aggregation. The present invention therefore relates to the use of TSP-1 in the study and development of treatment regimens, and to the direct use of TSP-1 in methods of treating or screening for thrombotic abnormalities.

Accordingly, in a first aspect the present invention provides a method for disrupting multimeric vWF, the method comprising exposing the multimeric vWF to TSP-1 or a functional fragment thereof.

In a second aspect the present invention provides a method for reducing platelet adhesion and aggregation in a subject, the method comprising administering to the subject an effective amount of TSP-1 or a functional fragment thereof.

In a third aspect the present invention provides a method for the treatment of a thrombotic disorder in a subject, the method comprising administering to the subject an effective amount of TSP-1 or a functional fragment thereof.

In a fourth aspect the present invention provides a method for reducing platelet adhesion and aggregation in a subject, the method comprising administering to the subject a nucleic acid molecule comprising a sequence encoding TSP-1 or a functional fragment thereof.

In a fifth aspect the present invention provides a method for the treatment of a thrombotic disorder in a subject, the method comprising administering to the subject a nucleic acid molecule comprising a sequence encoding TSP-1 or a functional fragment thereof.

In a sixth aspect the present invention provides a method for detecting a thrombotic disorder in a subject, the method comprising measuring the amount of TSP-1 in a biological sample derived from the subject.

In a preferred embodiment of the sixth aspect, the method comprises measuring the relative amount of TSP-1 to vWF. It will be appreciated that low or high relative levels of TSP-1 compared to those of a healthy subject are indicative of a potential thrombotic disorder in the subject.

In a further preferred embodiment of the sixth aspect, the sample is blood or a blood derived sample such as plasma.

In a seventh aspect the present invention provides a method of promoting platelet adhesion and aggregation in a subject, the method comprising administering to the subject an antagonist of TSP-1.

### BRIEF DESCRIPTION OF THE FIGURES

FIG. 1 Reduction in the average multimer size of vWF by conditioned medium from HMEC-1 cells. A Plasma (10 μl) from a patient with TTP was incubated with Hepes buffered saline containing 1 mM CaCl₂ and MgCl₂ (TTP, lane 1) or the conditioned media of HMEC-1 cells (+ECcm, lane 2) (90 μl) for 1 hour at 37° C. and aliquots of the reaction (10 μl) were resolved on 1% agarose gel electrophoresis. The vWF was transferred to PVDF membrane and Western blotted using peroxidase conjugated anti-vWF polyclonal antibodies. The bracket highlights the change in the proportion of large vWF multimers in the population. B Aliquots of the reactions described in part A were analyzed for vWF antigen levels and collagen binding affinity. The results are expressed as the ratio of the collagen binding activity and vWF antigen level. The bars and errors are the mean and SD of triplicate determinations.

FIG. 2 Purification of vWF reductase. A Thirty litres of conditioned medium from the human dermal microvascular endothelial cell line, HMEC-1, was collected, concentrated and applied to Heparin-Sepharose. The bound proteins were resolved with a linear NaCl gradient. vWF reductase activity eluted at −0.3M NaCl. B The peak of activity from the Heparin-Sepharose column was pooled, concentrated and gel filtered on Sephacryl S-300 HR. The vWF reductase activity resolved in the leading peak. C A sample (30 μl) of the pooled activity from the Sephacryl S-300 HR column was resolved on 4-15% SDS-PAGE under non-reducing (lane 1) or reducing (lane 2) conditions and silver stained. The vWF reductase had a molecular mass of ˜500 kDa which reduced to ˜170 kDa after reduction and alkylation of the protein. The positions of Mr markers are shown at left. D Purified human platelet TSP-1 (50 ng, lane 1) or a sample (30 μl) of the pooled activity from the Sephacryl S-300 HR column was resolved on 4-15% SDS-PAGE under non-reducing conditions, transferred to PVDF membrane and blotted with an anti-TSP-1 monoclonal antibody (HB8432). The positions of Mr markers are shown at left. E HMEC-1 conditioned medium (1 ml) was incubated alone or with an anti-TSP-1 or control anti-vWF monoclonal antibody (10 μg per ml) and Protein G-Sepharose beads (50 μl of packed beads) for 1 hour at 4° C. on a rotating wheel. The Sepharose beads were pelleted by centrifugation and the supernatant was assayed for vWF reductase activity. Plasma (10 μl) from a patient with TTP was incubated with the HMEC-1 conditioned medium supernatants (90 μl) for 1 hour at 37° C. and aliquots of the reaction were analyzed for vWF antigen levels and collagen binding affinity. The results are expressed as the ratio of the collagen binding activity and vWF antigen level. The bars and errors are the mean and SD of triplicate determinations.

FIG. 3 Reduction in the average multimer size of vWF by TSP-1 in vitro. A Plasma (10 μl) from a patient with TTP was incubated with Hepes buffered saline containing 1 mM CaCl, (TTP, lane 1) or purified platelet TSP-1 (1 μg per ml, lane 2) in the Hepes/CaCl₂ buffer (90 μl) for 1 hour at 37° C. and aliquots of the reaction (10 μl) were resolved on 1% agarose gel electrophoresis. The vWF was transferred to PVDF membrane and Western blotted using peroxidase conjugated anti-vWF polyclonal antibodies. The bracket highlights the change in the proportion of large vWF multimers in the population. B Plasma (10 μl) from a patient with TTP was incubated with HMEC-1 conditioned medium (+ECcm) or purified platelet TSP-1 (0.01 to 100 μg per ml) in Hepes buffered saline containing 1 mM CaCl₂ (90 μl) for 1 or 24 hour at 37° C. and aliquots of the reaction were analyzed for vWF antigen levels and collagen binding affinity. The results are expressed as the ratio of the collagen binding activity and vWF antigen level. The bars and errors are the mean and SD of triplicate determinations. C Interaction of TSP-1 with vWF. ELISA plate wells coated with purified human vWF and blocked with BSA were incubated with purified human TSP-1 (0 to 10 μg per ml) in Hepes buffered saline containing 1 mM CaCl₂ and no (open bars), 5 mM (hatched bars) or 20 mM (close bars) NEM for 30 minutes at room temperature. On one occasion wells not coated with vWF but blocked with BSA were incubated with 10 μg per ml TSP-1. The wells were washed with the Hepes buffer containing 1M NaCl to minimize non-covalent interactions and the bound TSP-1 was measured using an anti-TSP1 monoclonal antibody and peroxidase-conjugated secondary antibody. The bars and errors are the mean and SD of triplicate determinations. D Generation of new thiols in vWF by TSP-1. Purified human vWF (2 μg per ml) was incubated with Hepes buffered saline containing 1 mM CaCl₂ (Nil), HMEC-1 conditioned medium (CM) or purified human TSP-1 (0.01 to 100 μg per ml) in the Hepes/CaCl₂ buffer for 60 minutes at 37° C. The reactions were labeled with MPB (100 μM) and the unreacted MPB was quenched with GSH (200 μM). Aliquots of the reactions were incubated in ELISA plate wells coated with anti-vWF polyclonal antibodies and the adsorbed vWF was incubated with streptavidin peroxidase to measure the incorporated MPB. The bars and errors are the mean and SD of triplicate determinations.

### DETAILED DESCRIPTION OF THE PREFERRED EMBODIMENTS

The present invention is based on the surprising finding that thrombospondin-1 (TSP-1) is capable of reducing the multimer size of vWF by reducing disulphide linkages between vWF subunits. The present invention therefore relates to methods for disrupting and reducing the size of multimeric vWF and/or reducing platelet adhesion and aggregation in a subject. These methods are particularly useful in the treatment of thrombotic disorders.

The methods of the present invention comprise the use of thrombospondin-1 (TSP-1) or a functional fragment thereof.

The complete amino acid sequence of native TSP-1 is described in Lawler and Hynes, 1986.

The term “thrombospondin-1” or “TSP-1” as used herein is intended to encompass functional analogues and conjugates of TSP-1 that retain the ability of the native protein to reduce disulphide bonds in multimeric vWF.

The phrase “functional fragment thereof” as used herein is intended to encompass fragments of TSP-1 that retain the ability of the native protein to reduce disulphide bonds in multimeric vWF.

TSP-1 and analogues and functional fragments thereof can be either naturally occurring (that is to say, purified or isolated from a natural source) or synthetic (for example, by performing site-directed mutagenesis on the encoding DNA or by chemical synthesis of peptide fragments). It is thus apparent that TSP-1 as used in the present invention can be either naturally occurring or synthetic or recombinant.

Functional analogues may be polypeptides derived from TSP-1 in which deletions, insertions, additions or substitutions of amino acid residues are made. Amino acid sequence insertions include amino and/or carboxyl-terminal fusions ranging in length from one residue to polypeptides containing a hundred or more residues, as well as intrasequence insertions of single or multiple amino acid residues. Other insertional variants include the fusion of the N- or C-terminus of the proteins to an immunogenic polypeptide e.g. bacterial polypeptides such as betalactamase or an enzyme encoded by the E. coli trp locus, or yeast protein, bovine serum albumin, and chemotactic polypeptides. C-terminal fusions with proteins having a long half-life such as immunoglobulin constant regions (or other immunoglobulin regions), albumin, or ferritin, are included. Non-sequence modifications include acetylation, methylation, phosphorylation, carboxylation, or glycosylation.

It will be appreciated by those skilled in the art that a number of modifications may be made to the polypeptides and fragments of the present invention without deleteriously affecting the biological activity of the polypeptides or fragments. This may be achieved by various changes, such as sulfation, phosphorylation, nitration and halogenation; or by amino acid insertions, deletions and substitutions, either conservative or non-conservative (e.g. D-amino acids, desamino acids) in the peptide sequence where such changes do not substantially alter the overall biological activity of the peptide. Preferred substitutions are those which are conservative, i.e., wherein a residue is replaced by another of the same general type. As is well understood, naturally-occurring amino acids can be subclassified as acidic, basic, neutral and polar, or neutral and nonpolar. Furthermore, three of the encoded amino acids are aromatic. It is generally preferred that encoded peptides differing from the determined polypeptide contain substituted codons for amino acids which are from the same group as that of the amino acid replaced. Thus, in general, the basic amino acids Lys, Arg, and His are interchangeable; the acidic amino acids Asp and Glu are interchangeable; the neutral polar amino acids Ser, Thr, Cys, Gln, and Asn are interchangeable; the nonpolar aliphatic amino acids Gly, Ala, Val, Ile, and Leu are conservative with respect to each other (but because of size, Gly and Ala are more closely related and Val, Ile and Leu are more closely related), and the aromatic amino acids Phe. Trp and Tyr are interchangeable.

It should further be noted that if polypeptides are made synthetically, substitutions by amino acids which are not naturally encoded by DNA may also be made. For example, alternative residues include the omega amino acids of the formula NH₂(CH₂)ₙCOOH wherein n is 2-6. These are neutral, nonpolar amino acids, as are sarcosine, t-butyl alanine, t-butyl glycine, N-methyl isoleucine, and norleucine. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

The methods of treatment of the present invention involve administering an “effective amount” of TSP-1 or a functional fragment thereof to a subject. It will be appreciated that an “effective amount” of TSP-1 or a functional fragment thereof is an amount sufficient to disrupt multimeric vWF such that platelet adhesion and aggregation, or the potential for platelet adhesion and aggregation, in the subject is reduced. A person skilled in the art will be able to readily determine “an effective amount” on a case by case basis.

It will also be appreciated by those skilled in the art that TSP-1 or a functional fragment thereof may be introduced into a subject by administering a nucleic acid molecule comprising a sequence encoding TSP-1 or a functional fragment thereof.

The nucleic acid molecule may be in the form of DNA or RNA or a chimeric molecule comprising both DNA or RNA.

A nucleotide sequence encoding TSP-1 may be cloned into an expression vector where the sequence encoding the agent is operably linked with expression control elements. Expression control elements are well known in the art and include, for example, promoters, enhancers and appropriate start and stop codons.

A variety of methods can be used for introducing a nucleic acid encoding TSP-1 into a target cell in vivo. For example, the naked nucleic acid may be injected at the target site, may be encapsulated into liposomes, or may be introduced by way of a viral vector.

Direct injection of a nucleic acid molecule alone or encapsulated, for example, in cationic liposomes may be used for stable gene transfer of a nucleic acid encoding TSP-1 into non-dividing or dividing cells in vivo (Ulmer et al., Science 259:1745-1748 (1993)). In addition, the nucleic acid can be transferred into a variety of tissues in vivo using the particle bombardment method (Williams et al., Proc. Natl. Acad. Sci. U.S.A. 88:2726-2730 (1991)).

Viral vectors are useful for gene transfer of a nucleic acid molecules encoding TSP-1 into a specific cell type in vivo. Viruses are specialized infectious agents that can infect and propagate in specific cell types. This specificity for infecting particular cell types is especially suitable for targeting TSP-1 to selected cells in vivo. The selection of a viral vector will depend, in part, on the cell type to be targeted.

Specialized viral vectors are well known in the art that can target to specific cell types. Such vectors include, for example, recombinant adeno-associated viral vectors having general or tissue-specific promoters (Lebkowski et al. U.S. Pat. No. 5,354,678). Recombinant adeno-associated viral vectors have the added advantage that the recombinant virus can stably integrate into the chromatin of even quiescent non-proliferating cells (Lebkowski et al., Mol. Cell. Biol. 8:3988-3996 (1988)).

Viral vectors can be constructed to further control the type of cell that expresses the encoded TSP-1 by incorporating a tissue-specific promoter or enhancer into the vector (Dai et al., Proc. Natl. Acad. Sci. U.S.A. 89:10892-10895 (1992)).

Retroviral vectors are also suitable for the methods for delivering nucleic acid molecules encoding TSP-1 in vivo. Such vectors can be constructed either to function as infectious particles or as non-infectious particles that undergo only a single initial round of infection.

Receptor-mediated DNA delivery approaches also can be used to deliver a nucleic acid molecule encoding TSP-1 into a cell in a tissue-specific manner using a tissue-specific ligand or an antibody that is non-covalently complexed with the nucleic acid molecule via a bridging molecule (Curiel et al., Hum. Gene Ther. 3:147-154 (1992); Wu and Wu, J. Biol. Chem. 262:4429-4432 (1987)).

Gene transfer to obtain expression of TSP-1 in a subject also can be performed by, for example, ex vivo transfection of autologous cells. Suitable cells for such ex vivo transfection include blood cells since these cells are readily accessible for manipulation and reintroduction back into the subject by methods well known in the art.

Gene transfer through transfection of cells en vivo can be performed by a variety of methods, including, for example, calcium phosphate precipitation, diethyaminoethyl dextran, electroporation, lipofection, or viral infection. Such methods are well known in the art (see, for example, Sambrook et al., Molecular Cloning: A Laboratory Manual, Cold Springs Harbour Laboratory Press (1989)). Once the cells are transfected, they are then transplanted or grafted back into a subject to be treated. The cells once introduced into the body can produce the TSP-1, which can enter the circulation and inhibit platelet adhesion and aggregation at the site of the disease or condition.

The present invention also provides a method for promoting platelet adhesion and aggregation in a subject, the method comprising administering to the subject an antagonist of TSP-1. It will be appreciated that methods for promoting platelet adhesion and aggregation may be useful in conditions of excessive bleeding, such as von Willebrand's disease and haemophilia.

Methods for promoting platelet adhesion and aggregation may also be useful in cases where overdosage of an antiplatelet drug has resulted in excessive bleeding in a subject. In these cases, an antagonist of TSP-1 may be useful as an antidote to the antiplatelet drug therapy.

It would be well within the capabilities of a person skilled in the art to screen for antagonists of TSP-1. For example, an assay base on collagen binding affinity (Favoloro et al., 1991) as described herein is a useful indicator of average vWF multimer size. This assay may be used to screen for compounds that have the ability to interfere with the reduction of vWF multimers by TSP-1.

In the context of the present invention, the TSP-1 or functional fragments thereof or antagonists thereof maybe administered in the form of compositions comprising physiologically acceptable liquid, gel or solid diluents, adjuvants and excipients. The peptide compounds may be formulated into the compositions as neutral or salt forms. These compositions can be administered to animals for veterinary use, such as wild domestic animals, and clinical use in humans in a similar manner to other therapeutic agents.

Typically, such compositions are prepared as injectables, either as liquid solutions or suspensions. Solid forms suitable for solution in, or suspension in, liquid prior to injection may also be prepared. The preparation my also be emulsified.

The compositions are conventionally administered parenterally, by injection, for example, either subcutaneously or intravenously. Additional formulations which are suitable for other modes of administration include suppositories, intranasal aerosols, and, in some cases, oral formulations.

It will be appreciated that the methods of the present invention may be useful in the treatment of conditions such as the following:

progression of atherosclerosis;

cerebrovascular accidents such as transient ischaemic; completed stroke; and after carotid surgery;

acute myocardial infarction (primary and secondary);

angina;

occlusion of coronary artery bypass graft;

occlusion following percutaneous transluminal coronary angioplasty;

occlusion following coronary stenting;

vascular occlusion in peripheral arterial disease; and

venous thromboembolic disease following surgery, during pregnancy or during immobilisation.

The methods of the present invention also be useful in the treatment of small vessel diseases such as:

Glomerulonephritis;

thrombotic thrombocytopenic purpura;

the haemolytic uraemic syndrome; and

placental insufficiency and preeclampsia.

The methods of the present invention also be useful in the treatment of vascular syndromes and myeloproliferative diseases.

The methods of the present invention also be useful in the treatment or prevention of thrombosis formation in:

artificial/prosthetic vascular shunts and grafts;

prosthetic heart valves;

cardiopulmonary bypass procedures; and

haemoperfusion and haemodialysis.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

The present invention is further described by the following non-limiting examples.

### EXAMPLE 1

### Reduction in the average multimer size of vWF by endothelial cell conditioned medium

Incubation of plasma with the conditioned medium of macrovascular (HUVEC and BAEC) or microvascular endothelial cells (HDVEC) for 24 hours resulted in a decrease in the average multimer size of vWF (not shown). Specifically, the very large multimers were lost. The contribution of the endogenous vWF in the endothelial cell conditioned media to the multimer patterns was negligible. Incubation of plasma with BAECcm for a further 24 hours did not result in any further depolymerization of vWF. This result indicated that endothelial cells secreted a factor which caused limited depolymerization of vWF.

### EXAMPLE 2

### Secretion and reduction in the average multimer size of vWF by HUVEC

Confluent HUVEC's were washed with M199 without FCS and incubated in M199 for up to 8.5 hours. The HUVECS were stimulated to release vWF by adding 30 mM human α-thrombin after 0.5 hours. The thrombin was quenched after 2 hours incubation by adding 5 mM D-Phe-Pro-Arg-chloromethyl ketone. Samples of the conditioned medium were collected at discrete time intervals and analyzed for vWF by 1% agarose gel electrophoresis and Western blotting or vWF antigen levels and collagen binding affinity (not shown). vWF antigen levels were determined using ELISA by capturing the vWF with adsorbed anti-vWF polyclonal antibodies and detecting using peroxidase conjugated anti-vWF polyclonal antibodies (Favoloro et al., 1991). Collagen binding affinity was assessed by incubating the vWF in plastic wells coated with type I/III collagen for 1 hour and measuring the bound vWF using peroxidase conjugated anti-vWF antibodies (Favoloro et al., 1991). Large vWF multimers bind to collagen with up to ˜100-fold higher affinity than smaller multimers, therefore affinity for collagen is a useful indicator of average vWF multimer size.

It was apparent from the results that the average multimer size of the vWF secreted by HUVEC was being reduced with time of incubation. For instance, secretion of vWF antigen increased following addition of thrombin and plateaued at ˜3 hours incubation. In contrast, the average vWF multimer size measured by either agarose gel electrophoresis or collagen binding peaked at 3 hours and was significantly decreased thereafter.

### EXAMPLE 3

### Inhibitor profile of the vWF depolymerizing activity of BAEC conditioned medium

Heat treatment of BAECcm ablated the depolymerizing activity measured by either agarose gel electrophoresis or collagen binding, implying that the active factor was a protein. The depolymerizing activity was not inhibited by the serine proteinase inhibitors, 4-(2-aminoethyl)benzenesulfonylfluoride, aprotinin, or chymostatin; the serine and cysteine proteinase inhibitor, leupeptin; the aspartic proteinase inhibitor, pepstatin; or the metalloproteinase inhibitor, phosphoramidon. However, pretreatment of BAECcm with the thiol blocking reagents, iodoacetamide (IAM), N-ethylmaleimide (NEM) or E-64, inactivated the depolymerase. Pretreatment of BAECcm with EDTA also ablated activity. Incubation of vWF with IAM or NEM alone did not have any discernible effect on the average multimer size of vWF (not shown).

The Tyr842-Met843 peptide bond in the A2 domain of vWF is very slowly cleaved in plasma. This produces polypeptide fragments of 176 and 140 kDa. The pattern and quantity of vWF polypeptide fragments following treatment with BAECcm were compared with untreated vWF. There was no difference in the vWF fragment pattern in the control versus BAECcm treated vWF, despite the fact that the average multimer size of vWF was decreased.

The finding that the vWF depolymerizing activity of BAECcm was inactivated by heat and thiol blocking reagents, and that the reduction in vWF multimer size was not associated with discernible peptide bond cleavage, implied that depolymerization had involved reduction of the disulfide bonds that held the multimers together. This suggested that the depolymerase was a protein disulfide bond reductase. One consequence of reduction of disulfide bond(s) in vWF would have been the formation of cysteines or free thiols in the depolymerized vWF. This hypothesis was tested by measuring incorporation of a biotin-linked thiol reactive compound into depolymerized vWF.

### EXAMPLE 4

### Assay for reduction of vWF disulfide bond(s) in BAECcm

The biotin-linked maleimide, MPB, was used to measure reduction of vWF disulfide bond(s) in BAECcm. Purified plasma vWF (2 mg per ml) was incubated in BAECcm (0 to 50% of the reaction volume) and Hepes buffered saline for up to 60 minutes at 37° C. Free thiol(s) formed in vWF by reduction of disulfide bonds bond(s) were labelled with MPB (100 μM) and the unreacted MPB was quenched with GSH (200 μM). N-ethylmaleimide reacts rapidly with free thiols with a second order rate constant of ˜10⁴ M⁻¹s⁻¹ at pH 8 (Torchinski and Dixon, 1974). Therefore, the half life for reaction of free thiols in vWF with the maleimide moiety of MPB was anticipated to be in the order of a few seconds. Aliquots of the reactions containing MPB-labelled vWF were incubated in ELISA plate wells coated with anti-vWF polyclonal antibodies. The adsorbed vWF was incubated with streptavidin peroxidase to measure the incorporated MPB. The control for vWF labelling was incubation and labelling of BAECcm alone, prior to addition of vWF. This reaction represented the contribution to the assay of MPB labelling of endogenous vWF and/or other vWF binding proteins in the BAECcm. The plasma vWF was pretreated with N-ethylmaleimide to block any existing thiols in the purified protein.

There was negligible labelling of vWF by MPB when incubated with buffer alone. However, incubation of vWF with BAECcm resulted in readily measured incorporation of MPB (not shown). The signal after correction for the background contribution of BAECcm represented MPB labelling of fee thiol(s) in the purified vWF following reduction of vWF disulfide bond(s) in the BAECcm. The results employed a vWF concentration of 2 μg per ml in the reactions with BAECcm which gave an optimal signal to noise ratio. The signal to noise ratio was not improved when vWF concentrations of either 5 or 10 μg per ml were used. A strong signal was observed at 1 in 50 dilution of BAECcm, which approximately doubled when a 1 in 2 dilution of BAECcm was used. The reduction of disulfide bond(s) in vWF in BAECcm and labelling with MPB was rapid and occurred within 1 minute of incubation. The incorporation of MPB did not appreciably change from 1 to 60 minutes incubation.

The feasibility of the hypothesis that vWF multimer size was reduced by a reductase secreted by endothelial cells was tested by examining the effects of purified reductants on vWF multimer size.

### EXAMPLE 5

### Effect of purified reductants on the average multimer size of purified vWF

Incubation of purified vWF with the purified reductants, GSH (20 μM), PDI (10 nM), or thioredoxin (10 nM) for 24 hours at 37° C. resulted in reduction of the average multimer size of vWF (not shown). As observed with BAECcm, the large multimers were lost. Incubation for a further 24 hours did not result in any further depolymerization of vWF.

The effect of incubation of purified vWF with GSH, PDI, or GSH and PDI, for 24 hours at 37° C. on the affinity of vWF for collagen was analysed. Both of the reductants reduced the collagen affinity of vWF. The half-maximal effect of GSH on vWF collagen affinity was ˜20 μM. PDI at 10 nM reduced vWF collagen affinity to the same extent as 20 μM GSH. PDI and GSH in combination further reduced vWF collagen affinity. The half-maximal effect of GSH in the presence on PDI on vWF collagen affinity was similar to that of GSH alone, ˜20 μM.

PDI is on the surface of platelets and BAEC, although PDI was not detected in BAECcm. The possibility that trace levels of PDI was responsible for the vWF reductase activity in BAECcm was examined by incubating BAECcm with affinity purified anti-PDI polyclonal antibodies and testing for vWF reductase activity. Incubation of BAECcm with anti-PDI antibodies or pre-immune control IgG did not effect the vWF reductase activity of BAECcm. This result indicated that PDI was not the vWF reductase in BAECcm.

### EXAMPLE 6

### Effect of GSH on the vWF depolymerizing activity of BAECcm

To test whether exogenous GSH could augment vWF depolymerization by BAECcm, plasma vWF was incubated with BAECcm, or BAECcm and GSH (20 μM), and multimer size measured by either agarose gel electrophoresis or collagen binding.

Addition of GSH to BAECcm caused a small increase in vWF depolymerization over that by BAECcm alone, although BAECcm alone accounted for most of the depolymerization. Addition of GSH to BAECcm caused a significant reduction in the collagen affinity of vWF (p<0.05).

### EXAMPLE 7

### Properties of the vWF depolymerizing activity of BAECcm

BAECcm was incubated with either S-Sepharose, Q-Sepharose, heparin-Sepharose or activated thiol-agarose at a matrix:BAECcm ratio of 1:20 for 1 hour at 4° C. and the matrix sedimented by centrifugation. TTP plasma was incubated with the untreated or treated BAECcm for 24 hours at 37° C. and the average vWF multimer size measured be either agarose gel electrophoresis or collagen binding. It was apparent from the results that the vWF depolymerizing activity of BAECcm bound to S-Sepharose, heparin-Sepharose and activated thiol-agarose, but not to Q-Sepharose. The findings discussed in Examples 1 to 7 suggested that the vWF depolymerizing activity was a protein with an anionic pI that binds heparin and contains one or more reactive cysteines. The binding to activated thiol-agarose was in accordance with inactivation of the depolymerizing activity by thiol blocking reagents.

### EXAMPLE 8

### Reduction in the average multimer size of vWF by HMEC-1 cells

Plasma was obtained from a healthy volunteer and during therapeutic plasmapheresis of a 73 year old female with TTP. For two weeks prior to admission she had been treated with “triple therapy” for Helicobacter pylorii. On admission she had severe diarrhea, was confused and was in cardiac and renal failure (serum creatinine was 621 mol per l (60-111)). The hemoglobin was 99 g per l (120-153), white cell count 8.4×10⁹ per l (4-11) and platelet count 23×10⁹ per l (150-400). She responded to repeated plasmapheresis and dialysis and was discharged well.

TTP plasma was incubated with 20 mM Hepes, 0.14M NaCl, 1 mM CaCl₂, 1 mM MgCl₂, pH 7.4 buffer, conditioned media of HMEC-1 cells or the Hepes buffer containing TSP-1 for 1 or 24 hours at 37° C. Volumes and concentrations of reactants are indicated in the figure legends. Aliquots of the reactions were diluted 10-fold in the Hepes buffer and assayed for collagen binding affinity and vWF antigen as described by Favaloro et al. (1991). On some occasions, aliquots of the reactions were resolved on 1% agarose gel electrophoresis (Ruggeri and Zimmerman, 1980), transferred to PVDF membrane (DuPont NEN, Boston, Mass.), blotted with 2 μg per ml of horse radish peroxidase conjugated anti-vWF polyclonal antibodies (DAKO Corporation, Carpinteria, Calif.) and visualized using chemiluminescence (DuPont NEN, Boston, Mass.).

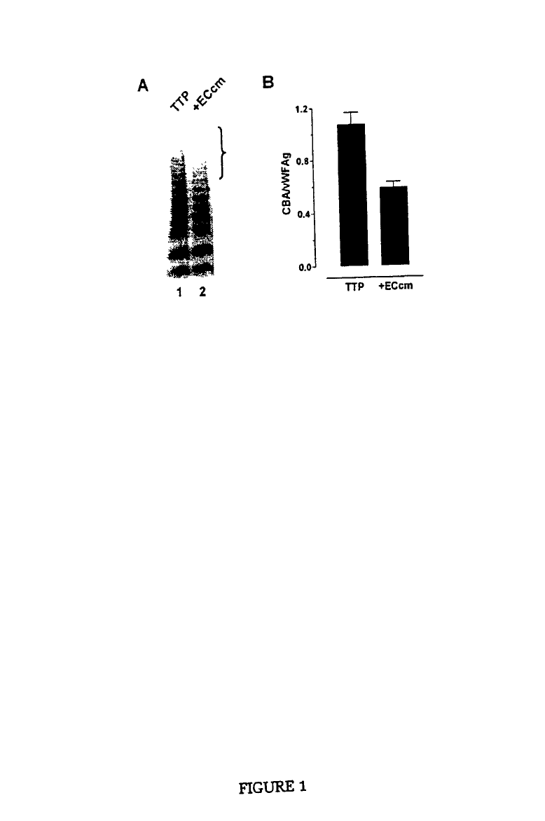

Incubation of plasma from a patient with TTP with the conditioned medium of the human dermal microvascular endothelial cell line, HMEC-1 (Ades et al., 1992), resulted in decrease in the average multimer size of vWF (FIG. 1A). Specifically, the very large multimers were lost (see bracket in FIG. 1A). There was negligible endogenous vWF in the HMEC-1 conditioned medium (not shown). The loss of the large vWF multimers upon incubation with HMEC-1 conditioned medium was reflected in decrease in affinity of the vWF for collagen (FIG. 1B). Affinity of vWF for collagen is an accurate and sensitive measure of average vWF multimer size (Favaloro et al., 1991; Siekmann et al.. 1998). Collagen binding was expressed relative to the vWF antigen level which takes into account any variation in the total vWF in the assays.

### EXAMPLE 9

### Purification of vWF reductase

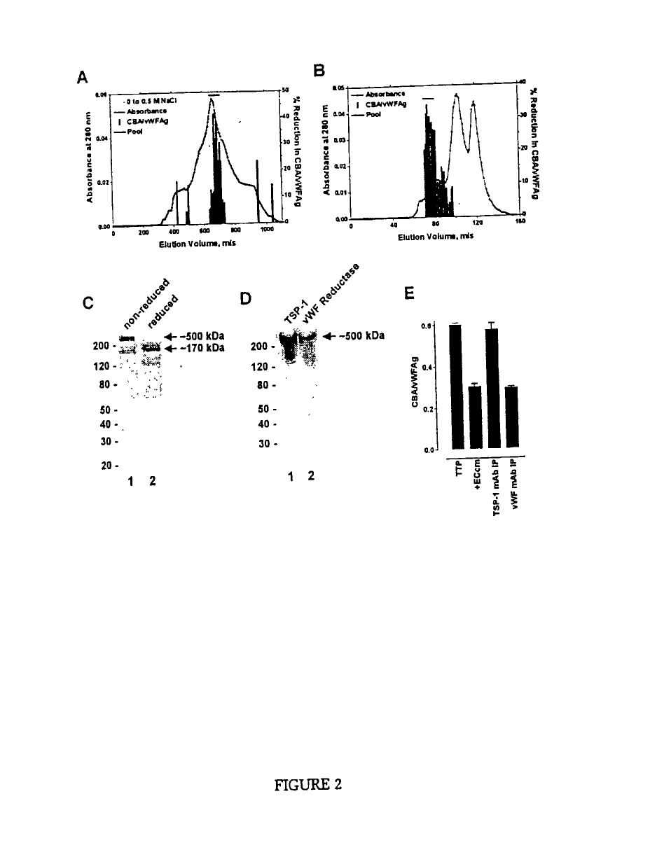

The reducing activity secreted by endothelial cells was associated with a protein with an anionic pI that binds heparin and contains reactive thiol(s) (see Example 7). Conditioned medium of HMEC-1 cells was prepared using Nunc Cell Factories. HMEC-1 (˜80,000 cells per cm² of cell factory area) were seeded into cell factories in MCDB-131 medium (Gibco BRL, Gaithersburg, Md.) containing 10 ng per ml EGF (Gibco BRL, Gaithersburg, Md.), 1 μg per ml hydrocortisone (Sigma, St. Louis, Mo.) and 10% fetal calf serum (Gibco BRL, Gaithersburg, Md.). When the cells were at 80% confluence they were washed twice with phosphate-buffered saline (Sigma, St. Louis, Mo.) and incubated with serum-free MCDB-131 medium at 37° C., 5% CO₂ for 30 hours. The conditioned medium was collected, centrifuged at 1200 g for 10 minutes and passed through a 0.2 μm Millipore filter to remove detached cells and cellular debris, and stored at −20° C. Thirty litres of conditioned medium (910 mg) was concentrated to 350 mls using a Amicon spiral-wound concentrator with a 10 kDa cutoff membrane. The proteinase inhibitors, leupeptin (10 μM), phenylmethylsulfonyl fluoride (1 mM) and soybean trypsin inhibitor (10 μg per ml) were added to the concentrated medium to minimize proteolytic degradation of the vWF reductase. Leupeptin, phenylmethylsulfonyl fluoride, soybean trypsin inhibitor and D-Phe-Pro-Arg-chloromethyl ketone were from Calbiochem, Alexandria, NSW. The concentrated medium was applied to a 150 ml column of Heparin-Sepharose (2.5'30 cm) equilibrated with 20 mM Hepes, 1 mM CaCl₂, 1 mM MgCl₂, 0.02% NaN₃, pH 7.4 buffer. The column was washed with 3 bed volumes of the Hepes buffer at a flow rate of 0.5 ml per min to elute unbound proteins and developed with a 2.2 L linear NaCl gradient from 0 to 1M in the Hepes buffer. vWF reductase activity eluted at ˜0.3M NaCl (˜700 mls) (FIG. 2A). The fractions containing vWF reductase activity (˜45 mls, ˜3 mg) were concentrated to 5 ml, dialyzed against 20 mM Hepes, 0.05M NaCl, 1 mM CaCl₂. 1 mM MgCl₂, 0.02% NaN₃, pH 7.4 buffer, and applied to a 210 ml column of Sephacryl S-300 HR (1.5×120 cm) (Pharmacia, Uppsala, Sweden) at a flow rate of 0.5 ml per minute. The vWF reductase activity resolved in the leading peak (˜0.1 mg) (FIG. 2B).

The enzyme had a molecular mass of ˜500 kDa on SDS-PAGE (Laemmli, 1970) which reduced to ˜170 kDa after reduction with 20 mM dithiothreitol and alkylation with 40 mM iodoacetamide (FIG. 2C). This subunit structure was very similar to that of thrombospondin-1 (TSP-1), which is a homotrimer of ˜170 kDa subunits that is secreted by endothelial cells (Mosher et al., 1982) and functions in cell-cell and cell-matrix interactions (Lawler, 2000). The enzyme was resolved on SDS-PAGE, transferred to PVDF membrane and blotted with a murine anti-TSP-1 monoclonal antibody (used at 2 μg per ml). The murine anti-TSP-1 monoclonal antibody hybridoma cell line, HB8432 (Jaffe et al., 1983), was obtained from ATCC, Rockville, Md. Antibody was produced in ascites and purified using Protein G-Sepharose (Pharmacia, Uppsala, Sweden). The HB8432 antibody was blotted with rabbit anti-mouse horseradish peroxidase conjugated antibodies (Dako Corporation, Carpinteria, Calif.) (used at 1:2000 dilution) and detected by chemiluminescence (DuPont NEN, Boston, Mass.). The HMEC-1 protein was recognized by the anti-TSP-1 monoclonal antibody in Western blot (FIG. 2D) and immunoprecipitation of TSP-1 from HMEC-1 conditioned medium accounted for all the vWF reductase activity in the medium (FIG. 2E).

### EXAMPLE 10

### Reduction in the average multimer size of vWF by TSP-1 in vitro

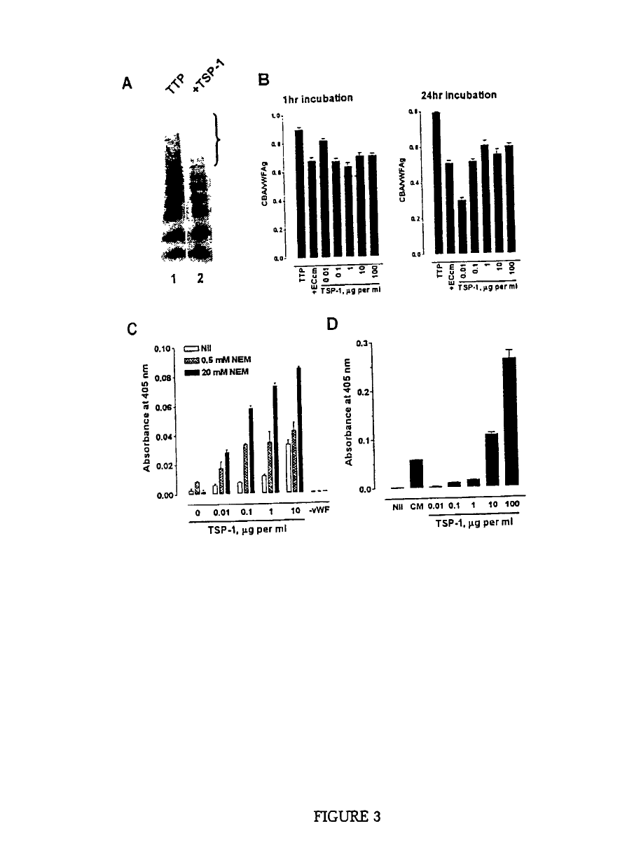

TSP-1 is a major component of platelet α-granules which is secreted upon platelet activation and aggregation. TSP-1 was purified to homogeneity from pooled outdated human platelet concentrates (Murphy-Ullrich and Mosher. 1985; Hogg et al., 1997) and tested for vWF reductase activity. Buffers containing 0.1 mM CaCl₂ were used throughout the chromatographic purification of TSP1. Platelet TSP-1 reduced the average multimer size of vWF in buffer (not shown) or in plasma (FIG. 3A). In particular, the very large multimers were lost (see bracket in FIG. 3A). Decrease in vWF multimer size was associated with decrease in affinity of vWF for collagen (FIG. 3B). TSP-1-mediated reduction in vWF multimer size was concentration- and time-dependent (FIG. 3B). Three different preparations of platelet TSP-1 had the same vWF reducing activity (not shown). It is noteworthy that the molar ratio of TSP-1 to vWF influenced the extent of reduction in vWF multimer size (FIG. 3B). The vWF reductase activity in endothelial cell conditioned medium is inhibited by EDTA. In accordance with this observation, chelation of Ca²⁺ with EDTA ablated the vWF reductase activity of TSP-1 (not shown).

The first step in reduction of a disulfide bond is nucleophilic attack on the substrate disulfide bond by a reductant thiol which results in formation of a disulfide-linked complex between the substrate and the reductant. Release of the reductant from the complex requires nucleophilic attack on the disulfide linkage by another thiol, usually of the reductant. We reasoned that N-ethylmaleimide (NEM, Sigma, St. Louis, Mo.) might trap intermediate covalent complexes of TSP-1 and vWF by blocking the TSP-1 thiol responsible for separating TSP-1 and vWF. Maleimides react rapidly and specifically with cysteine thiols at neutral pH. This prediction was tested by measuring formation of salt-resistant complexes between TSP-1 and vWF and the effect of the thiol-blocking reagent, NEM, on complex formation.

Purified vWF from human plasma was obtained from the Baker Medical Research Institute, Melbourne and was purified according to Booth et at. (1984). The vWF (100 μl of 5 μg per ml in 0.1M NaHCO3, pH 8.3 buffer) was adsorbed to Nunc PolySorp 96 well plates overnight at 4° C. in a humid environment. Wells were washed once with Hepes buffered saline, non-specific binding sites blocked by adding 200 μl of 2% BSA in Hepes buffered saline and incubating for 90 minutes at 37° C., and then washed two times with Hepes buffered saline. Coated wells were incubated with TSP-1 (0 to 10 μg per ml) in 20 mM Hepes, 0.14M NaCl, 1 mM CaCl₂, 1 mM MgCl₂, pH 7.4 buffer containing 0, 5 or 20 mM NEM for 30 minutes at room temperature with orbital shaking. On one occasion wells not coated with vWF but blocked with BSA were incubated with 10 μg per ml TSP-1. The wells were washed 4 times with the Hepes buffer containing 1M NaCl to minimize non-covalent interactions between TSP-1 and vWF and 100 μl of 10 μg per ml anti-TSP1 monoclonal antibody was added and incubated for 30 minutes at room temperature with orbital shaking. The wells were washed 3 times with the Hepes buffer and 100 μl of 1:1000 dilution of rabbit anti-mouse peroxidase-conjugated antibodies was added and incubated for 30 minutes at room temperature with orbital shaking. Wells were washed three times with Hepes buffer and the peroxidase detected as described previously (Favaloro et al., 1991). Up to 8-fold more TSP-1 bound to vWF in the presence of NEM (FIG. 3C). Complex formation increased with increasing NEM concentration. There was no binding of TSP-1 to wells not coated with vWF.

Reduction of disulfide bonds in vWF was anticipated to result in a net increase in free thiols in the vWF population. The biotin-linked maleimide, 3-(N-maleimidylpropionyl)biocytin (MPB, Molecular Probes Incorporated, Eugene. Oreg.), was used to measure the formation of new thiols in vWF upon incubation with TSP-1. Briefly, purified human vWF (2 μg per ml) was incubated with 20 mM Hepes, 0.14M NaCl, 1 mM CaCl₂, 1 mM MgCl₂, pH 7.4 buffer, HMEC-1 conditioned medium or the Hepes buffer containing purified human TSP-1 or peptides for 60 minutes at 37° C. Free thiol(s) formed in vWF by reduction of disulfide bond(s) were labeled with MPB (100 μM) for 10 minutes at 37° C. and the unreacted MPB was quenched with reduced glutathione (GSH, Sigma, St. Louis, Mo.) (200 μM) for 10 minutes at 37° C. The MPB-labeled vWF was incubated in ELISA plate wells coated with anti-human vWF polyclonal antibodies and the biotin label was detected using StreptABComplex/HRP (DAKO Corporation, Carpinteria, Calif.).

There was negligible existing free thiols in the purified vWF. Incubation of vWF with either HMEC-1 conditioned medium or purified TSP-1 resulted in incorporation of MPB into vWF and the incorporation increased with increasing TSP-1 concentration (FIG. 3D). Chelation of Ca²⁺ in the reactions with EDTA ablated TSP-1-catalysed formation of free thiols in vWF (not shown).

### EXAMPLE 11

### Reduction in the average multimer size of vWF by TSP-1 in vivo.

The ability of TSP-1 to reduce the average multimer size of vWF in vivo was examined by administering TSP-1 to mice via intraperitoneal injection and measuring the consequence for the average multimer size of plasma vWF. Balb c mice (7 to 9 weeks of age) were administered either buffer vehicle (n=4) or 1 mg per kg purified human platelet TSP-1 (n=4) via 0.1 ml intraperitoneal injections. There were 2 male and 2 female mice in each group. Blood was collected by cardiac puncture into EDTA 24 hours later and the plasma was analyzed in triplicate for vWF antigen levels and collagen binding affinity. The average multimer size of the plasma vWF was significantly lower (p<0.05) in mice treated with TSP-1 (CBA/vWFAg=0.83±0.05) than with buffer vehicle (CBA/vWFAg=1.65±0.32).

Publications referred to above are incorporated herein in their entirety by this reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Ades, E. W., F. J. Candal, R. A. Swerlick, V. G. George, S. Summers, D. C. Bosse, and T. J. Lawley. 1992. HMEC-1: establishment of an immortalized human microvascular endothelial cell line. J. Invest. Dermatol. 99:683-690.

Booth, W. J., F. H. Furby, M. C. Berndt, and P. A. Castaldi. 1984. Factor VIII/von Willebrand factor has potent lectin activity. Biochem. Biophys. Res. Commun. 118:495-501.

Dong, Z., R. S. Thoma, D. L. Crimmins, D. W. McCourt, E. A. Tuley, and J. E. Sadler. 1994. Disulfide bonds required to assemble functional von Willebrand factor multimers. J. Biol. Chem. 269:6753-6758.

Favaloro, E. J., L. Grispo, T. Exner, and J. Koutts. 1991. Development of a simple collagen based ELISA assay aids in the diagnosis of, and permits sensitive discrimination between Type I and Type II, von Willebrand's disease. Blood Coag. Fibrinol. 2:285-291.

Furlan, M. 1996. von Willebrand factor: molecular size and functional activity. Ann. Hematol. 72:341-348.

Furlan, M., B. A. Perret, and E. A. Beck. 1979. Studies on factor VIII-related protein. III. Size distribution and carbohydrate content of human and bovine factor VIII. Biochim. Biophys. Acta 579:325-333.

Hogg, P. J., K. A. Hotchkiss, B. M. Jiménez, P. Stathakis, and C. N. Chesterman. 1997. Interaction of platelet-derived growth factor with thrombospondin 1: dependence on the disulfide-bond arrangement in thrombospondin 1. Biochem. J. 326:709-716.

Jaffe, E. A., J. T. Ruggiero, L. L. Leung, M. J. Doyle, P. J. McKeown-Longo, and D. F. Mosher. 1983. Cultured fibroblasts synthesize and secrete thrombospondin and incorporate it into extracellular matrix. Proc. Natl. Acad. Sci. U.S.A. 80:998-1002.

Laemmli, U. K. 1970. Cleavage of structural proteins during assembly of the head of bacteriophage T4. Nature 227:680-685.

Lawler, J. 2000. The functions of thrombospondin-1 and -2. Curr. Opin. Cell Biol. 12:634-640.

Lawler, J. and Hynes, R. O. 1986. The structure of human thrombospondin, an adhesive glycoprotein with muoltiple calcium-binding sites and homologies with several different proteins. The Journal of Cell Biology 103:1635-1648.

Moake, J. L. 1997. Studies on the pathophysiology of thrombotic thrombocytopenic purpura. Sem. Hematol. 34:83-89.

Moake, J. L., N. A. Turner, N. A. Stathopoulos, L. H. Nolasco, and J. D. Hellums. 1989. Involvement of large plasma van Willebrand factor (vWF) multimers and unusually large vWF forms derived from endothelial cells in shear stress-induced platelet aggregation. J. Clin. Invest. 78:1456-1461.

Mosher, D. F., M. J. Doyle and E. A. Jaffe. 1982. Synthesis and secretion of thrombospondin by cultured human endothelial cells. J. Cell Biol. 93:343-348.

Murphy-Ullrich, J. E., and D. F. Mosher. 1985. Localization of thrombospondin in clots formed in situ. Blood 66:1098-1104.

Ruggeri, Z. M., and T. S. Zimmerman. 1980. Variant van Willebrand's disease: Characterization of two subtypes by analysis of multimer composition of factor VIII/von Willebrand factor in plasma and platelets. J. Clin. Invest. 65:1318-1325.

Siekmann, J., P. L. Turecek, and H. P. Schwarz. 1998. The determination of von Willebrand factor activity by collagen binding assay. Haemophilia 4:15-24.

1. A method for disrupting multimeric vWF, the method comprising exposing the multimeric vWF to thrombospondin-1 (TSP-1) or a functional fragment thereof.

2. A method for reducing platelet adhesion and aggregation in a subject, the method comprising administering to the subject an effective amount of TSP-1 or a functional fragment thereof.

3. A method for the treatment of a thrombotic disorder in a subject, the method comprising administering to the subject an effective amount of TSP-1 or a functional fragment thereof.

4. A method for reducing platelet adhesion and aggregation in a subject, the method comprising administering to the subject a nucleic acid molecule comprising a sequence encoding TSP-1 or a functional fragment thereof.

5. A method for the treatment of a thrombotic disorder in a subject, the method comprising administering to the subject a nucleic acid molecule comprising a sequence encoding TSP-1 or a functional fragment thereof.

6. A method for diagnosing the potential for a thrombotic disorder in a subject, the method comprising measuring the amount of TSP-1 in a biological sample derived from the subject.

7. A method as claimed in claim 6 which comprises measuring the relative amount of TSP-1 to vWF.

8. A method as claimed in claim 6 or claim 7 in which the sample is blood or a blood derived sample such as plasma.

9. A method for promoting platelet adhesion and aggregation in a subject, the method comprising administering to the subject an antagonist of TSP-1.

10. A method for reversing the effects of an overdose of an antiplatelet agent in a subject, the method comprising administering to the subject an antagonist of TSP-1.
